(19) United States
(12) Patent Application Publication
(10) Pub. No.: US 2018/0296497 A1
(43) Pub. Date: Oct. 18, 2018

(54) Method and Articles for Inhibiting Bladder Contractions

(71) Applicant: Avent, Inc., Alpharetta, GA (US)

(72) Inventors: Eric A. Schepis, Alpharetta, GA (US); Nicole Y. Dumas, Woodstock, GA (US)

(21) Appl. No.: 15/519,958

(22) PCT Filed: Oct. 28, 2015
(86) PCT No.: PCT/US2015/057711

§ 371 Date: Apr. 18, 2017

Related U.S. Application Data
(60) Provisional application No. 62/073,315, filed on Oct. 31, 2014.

(51) Int. Cl. A61K 9/70; A41B 9/00; A61J 1/00; A61K 31/045; A61K 31/165; A61K 9/00

(52) U.S. Cl. CPC A61K 9/7023; A41B 9/001; A61J 1/00; A41B 2400/32; A61K 31/165; A61K 9/0014; A61K 31/045

(57) ABSTRACT

A method and article for alleviating a subject's urge to urinate is described. The article includes a support and a composition with a stimulation agent such as menthol, menthol derivatives, capsaicin, or a combination thereof. The support may be in the form of a wipe, patch or pant. In human subjects, the composition is applied to select dermatomes for a desired period of time.

### RELATED APPLICATIONS

The present application claims priority to U.S. Provisional Application Ser. No. 62/073,315, filed on Oct. 31, 2014, which is incorporated herein in its entirety by reference thereto.

### FIELD OF THE INVENTION

This disclosure relates to the use of stimulus to alleviate the urge to urinate. In particular, a composition with a stimulation agent is applied to intact skin to activate sensory receptors and elicit bladder inhibition.

### BACKGROUND

Overactive bladder is a condition in which involuntary bladder contractions occur during bladder filling despite a person's attempt to suppress them. It causes symptoms such as urinary frequency and nocturia. Urge incontinence, which is involuntary leakage accompanied by urgency, may also occur.

The prevalence of overactive bladder and urinary incontinence and their impact on quality-of-life is substantial, necessitating better treatment options. Approximately 16.5% of US and European populations have the symptoms of overactive bladder: 49 MM persons in Europe and 34 MM persons in the United States. Overactive bladder is thought to have a greater impact on quality-of-life than type II diabetes, and its financial impacts are greater than both breast cancer ($12.7 billion) and osteoporosis treatments ($13.8 billion). Further, urinary incontinence affects 17-29.5 MM persons in the United States. About 50% of American women experience urinary incontinence during their lifetime.

Various treatments are available to mitigate overactive bladder in humans. Milder treatment options include lifestyle changes, bladder training and pelvic floor exercises (i.e. kegel exercises). These options are designed to train the bladder and support musculature to better hold urine. Although these methods may help the person suffering from overactive bladder, they are time consuming and are often unsuccessful. Other treatment options include medication, surgery and neuromodulation. Medication may not be desirable for some patients because of contraindications or lack of compliance. Surgery is reserved for persons that are severely affected by urge incontinence because it poses possible complications such as blood clots, bowel obstruction, infection and pneumonia. Neuromodulation treatments to mitigate overactive bladder are showing promise and are becoming more popular.

Neuromodulation technologies use electrical stimulation to modulate nerves that are positioned deep beneath the skin's surface. The systems deliver electrical stimulation to the sacral, tibial or pudendal nerves to modulate micturition (i.e. spinal) reflexes that are responsible for voiding. Stimulation is delivered to these nerves either directly (i.e. invasively) or indirectly, and act on different parts of the reflex pathway, yielding different outcomes. They have effectively treated persons with overactive bladder, including persons who have failed pharmacological therapies. These technologies require regulatory approval and must be used under clinical supervision.

There remains a need for a safe, effective and non-invasive method to mitigate the symptoms of overactive bladder that can be self-administered. There further remains a need for a method that does not consist of an oral medication, is available without prescription, and is affordable.

### SUMMARY

Disclosed is a kit for the alleviation of a subject's urge to urinate. The kit includes a support having a body-facing surface and a garment-facing surface. A composition comprising a stimulation agent selected from the group consisting of menthol, a menthol derivative, capsaicin, or a combination thereof is disposed onto or included for disposition by the subject onto an area of the body-facing surface. The stimulation agent is in an amount that can effectively modulate sensory receptors in cutaneous nerves or nerve branches to temporarily alleviate the urge to urinate. Included in the kit is a set of instructions regarding the placement of the composition onto the subject's buttock. The support may take various forms, including a wipe and a patch.

In another aspect is a method for alleviating a subject's urge to urinate. The method include the steps of: providing the subject with a support having a body-facing surface and a composition thereon, the composition comprising a stimulation agent selected from the group consisting of menthol, menthol derivatives, capsaicin, or a combination thereof, wherein the stimulation agent is present in an amount that can effectively modulate sensory receptors in cutaneous nerves or nerve branches to temporarily alleviate the urge to urinate; and providing instruction as to the placement of the support on a target area of the subject. The target area of the subject may be an area covering a portion of the subject's S1-S4 dermatomes, and in particular, the S3 dermatome. The target area may be about 36 to 42 cm2, and be located on the subject's buttock.

In yet another aspect of the disclosure is a disposable pant having a body-facing surface and an opposite exterior surface, a front side, a back side and a crotch region. Outside of the crotch region, the body-facing surface of the back side has a composition disposed thereon. The composition may include menthol, menthol derivatives, capsaicin, or a combination thereof. The disposable pant may be made from a nonwoven fabric and/or an elastomeric fabric. It may further include a liquid impermeable support between the composition and the body-facing surface of the back side.

These and other aspects of the present disclosure will become apparent upon reference to the following detailed description.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 is a schematic view of one example of a kit according to the present disclosure;

FIG. 2A is a cross-sectional view of one embodiment of a patch according to the present disclosure; FIG. 2B is a cross-sectional view of another embodiment of a patch according to the present disclosure;

FIG. 3 is a side elevation of a subject in which the S1-S4 dermatomes are illustrated;

FIG. 4 is a is a rear elevation of a subject in which the S1-S4 dermatomes are illustrated;

FIG. 5 is a rear elevation of a subject wearing the patch of FIG. 4;

FIG. 6 is a side perspective view of one embodiment of a carrying case for containing the patch of the present disclosure, in a partially zipped state; and

FIG. 7 is a rear perspective view of one embodiment of a pant according to the present disclosure.

### DEFINITIONS

“Disposable”, as used herein, means that, while the patches of the present disclosure may be stored in a container and reapplied to the user's body, they are intended to be thrown away, i.e., deposited in a suitable trash receptacle, after the stimulation agent has been fully expended.

“Rigid”, as used herein, means the property of a material wherein the material may be flexible, yet is substantially stiff and unyielding, and which does not form fold lines in response to gravitational pull or other modest forces.

“Intact skin”, as used herein, refers to skin that is sound, unbroken and uninjured, or not altered in any meaningful way.

Generally “subjects” are “mammals” or “mammalian,” where these terms are used broadly to describe organisms which are within the class mammalia, including the orders carnivore (e.g., dogs and cats), and primates (e.g., humans, chimpanzees, and monkeys). While humans are referred to in many embodiments of the disclosure, other mammals may benefit from the method of the present disclosure with minor modifications.

“Nonwoven fabrics” are a class of fabrics produced by attaching fibers (e.g. such as by chemical or mechanical means), or both. The nonwoven fabric is made by mechanical, chemical, thermal, or solvent means, or with an adhesive, or any combination of these, and is distinct from woven, knitted or tufted materials. Nonwoven fabrics may be made from synthetic thermoplastic polymers or natural polymers such as cellulose.

### DETAILED DESCRIPTION

It is to be understood by one of ordinary skill in the art that the present discussion is a description of exemplary aspects of the present invention only, and is not intended as limiting the broader aspects of the present invention.

The present disclosure is directed to a method for acutely reducing or eliminating the urge to urinate. The present disclosure further includes a kit for carrying out the method described herein.

The urge to void can be reduced or abolished by selective stimulation of sensory receptors that are located within the skin. A sensory receptor is an organ specialized to transduce various types of energy (i.e. mechanical and thermal) into neural signals. The neural signals are transmitted through cutaneous peripheral nerves as action potentials (or impulses) to the spinal cord and drive spinal reflexes. In turn, the reflexes inhibit bladder contractions and sensations of bladder fullness. The sensory receptors capable of driving the urinary-bladder ensemble are paired with the larger sensory fibers (i.e. Aβ, Aδ) that carry information about touch, pressure, cold and some pain to neurons in the lumbar and sacral spinal cord (e.g. neurological segments: L1-L3, S1-S4). The skin regions that are innervated by our targeted receptors and neural pathways cover the lower back, buttocks and genitals. The cutaneous nerves that enable bladder inhibition and host the targeted sensory receptors include: 1. Superior cluneal nerve (arise from L1, L2, L3); 2. Medial cluneal nerve (arise from S1, S2, S3); 3. Inferior cluneal nerve (posterior cutaneous femoral nerve; arises from S1, S2, S3); 4. Perforating cutaneous nerve (arises from S1, S2, S3), and 5. Pudendal nerve (arises from S2, S3, S4). The most effective stimulation site includes sensory receptors that transmit to the S2, S3 and S4 neurological segments.

The method of the present disclosure provides great freedom to those suffering from overactive bladder, allowing them to sleep through the night, shop, golf, enjoy a movie, drive long distances, and many other activities, without the urgent need to urinate. Additional uses of the method and corresponding articles described herein include treating the symptoms of overactive bladder in persons suffering from spinal cord injury (in part to mitigate or delay autonomic dysreflexia), multiple sclerosis, benign prostatic hyperplasia, diabetes, Parkinson's disease, stroke, and other neurological and urinary diseases. It is contemplated that persons that do not have overactive bladder may even want to use the method of the present disclosure so they may postpone urinating, such as during a long drive or sporting event. It may also be used for potty training.

In one aspect of the present disclosure, topical stimulation compositions are provided. These compositions contain a physiological stimulation agent that causes the sensation of cooling, actual cooling and/or mild irritation. For instance, in one embodiment, a topical patch may be made from an adhesive gel composition that is present on a support, where the adhesive gel composition includes the stimulation agent, a water-soluble polymer gel, water, and a water holding agent. In practice, the topical stimulation composition is applied to the intact skin of a subject, such as by employing the patch, and maintained at the site of application for a desired period of time. The stimulation composition is removed from the skin in order for urination to occur.

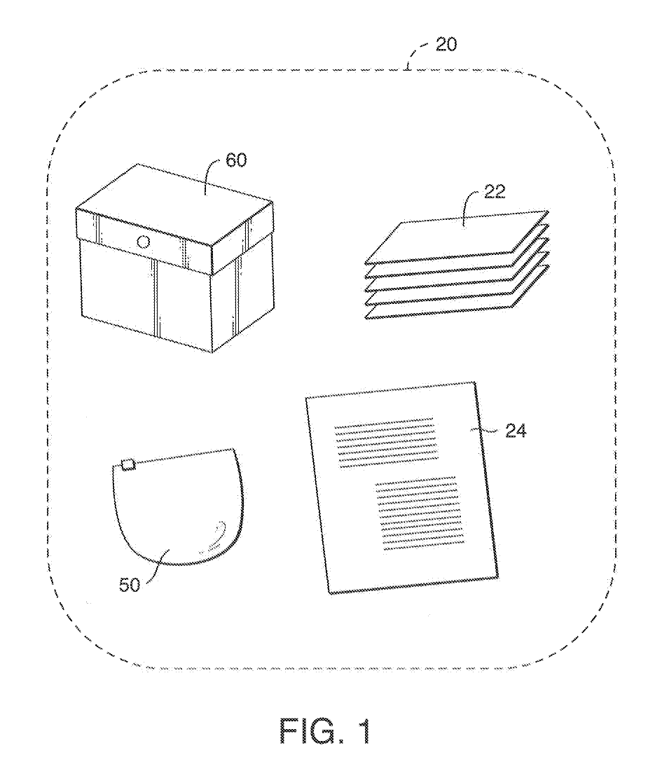

The topical stimulation composition, typically preapplied to a patch, may be included in an overactive bladder treatment kit shown in FIG. 1 as kit 20. In one embodiment, kit 20 may include at least one topical stimulation patch 22 and a set of instructions 24 detailing the method of using patch 22. A carrying case 50 may be included for discretely carrying patches 22 in a pocket or purse. A container 60 for dispensing more than one patch 22 may also be included.

In another embodiment (not shown) the topical stimulation composition is supplied to the user in the form of a water soluble and spreadable gel, cream, lotion or the like. Wipes, such as those used to clean the hands or body, may be provided so the subject may remove the gel, cream, lotion or the like from the body so urination can occur.

The stimulation patches 22 of the kits 20 are typically present in individual pouches or analogous packages, to preserve the composition of the patches until use. The kits 20 also generally include instructions 24 detailing how to use the patches, and which include information about where to apply the patch, application schedules, storage, etc.

The instructions 24 are generally recorded on a suitable recording medium. As seen in FIG. 1, for example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments not shown, the instructions are present as an electronic storage data file present on a suitable computer readable storage medium, e.g. CD-ROM, diskette, etc.

Each of the kit elements are described in detail below.

### Stimulation Patch

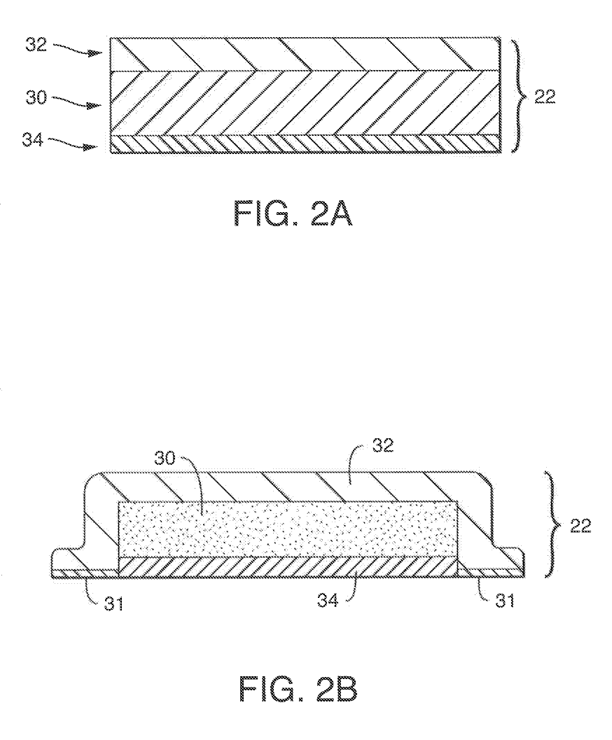

According to one aspect of the present disclosure and shown in FIG. 2, a topical patch 22 may include a gel adhesive base 30 present on a support 32. Each of these components is now described in greater detail.

The gel adhesive base 30, which serves as a retaining layer, is made up of the stimulation agent that is present in, e.g., dissolved in or dispersed in, the adhesive gel base. A “physiological stimulation agent” is an agent that, when contacted with skin of a subject, imparts a cooling sensation or effect, or skin irritation to the subject.

Representative physiological cooling agents of interest generally include menthol and various menthol derivatives including menthyl lactate, menthoxypropandiol, menthone glycerine acetal, and isopulegol. Some commercially available agents include: FRESCOLAT MGA, and FRESCOLAT ML (Haarmann & Reimer, Australia); COOLACT 38D, COOLACT P, and COOLACT 10 (Lipo Chemicals Company); and the like.

A representative physiological stimulation agent that irritates generally includes capsaicin.

The amount of stimulation agent that is present in the adhesive gel base 30 is an effective amount sufficient to administer to a targeted skin surface of a subject. Desirably, the stimulation agent will have a desired effect for up to 3-4 hours as long as it is in direct contact with the skin.

The adhesive gel base that includes the stimulation agent, as described above, is made up of a water-soluble high molecular weight substance, water and a water-retaining agent. In certain embodiments, the adhesive gel base 30 may further include a cosolvent, e.g., an organic cosolvent.

Water-soluble high molecular weight substances of interest include water-soluble polymers, where polymers of interest include, but are not limited to: gelatin, starch, agar, mannan, alginic acid, polyacrylic acid, sodium polyacrylate, dextrin, methylcellulose, sodium methylcellulose, hydroxypropylcellulose, sodium carboxymethylcellulose, cellulose gum, carboxyvinyl polymer, polyvinyl alcohol, polyvinylpyrrolidone, Arabia gum, acacia, tragacanth gum, karaya gum, and starch acrylate copolymer or other starch sodium acrylate graft copolymers. Metallic salts of these, as well as the products of cross-linking these by means of organic or inorganic cross-linking agents, are also of interest. These water-soluble polymers can be used to bring out the properties and characteristics of the other materials used in the adhesive gel composition, and in practice can be used alone or in combinations of two or more.

While any convenient water may be employed as the water component, of interest are distilled water or ion-exchange water or the like. The amount of water present in the adhesive gel base 30 is sufficient to impart the desired physical properties thereto. Desirably, the water content is such that the horny or keratinized layer of the skin does not swell significantly as it is not necessary for the stimulation agent to penetrate the skin.

The water-retaining agent or water-holding agent of the adhesive gel base 30 is any agent that is capable of at least diminishing the volatilization of water contained in the adhesive gel base so that the water content in the adhesive gel base is maintained at least a substantially constant, if not constant, level during storage and use of the preparation. One or more water-retaining agents may be employed in the subject compositions. Examples of suitable water-retaining agents include, but are not limited to: one or more types of polyvalent or polyhydric or sugars or alcohols, such as glycerin, sorbitol, propylene glycol, diethylene glycol, 1,3-butylene glycol, and ethylene glycol, and the like.

In addition, the gel base compositions may also include a cosolvent, where the cosolvent is generally an organic cosolvent. Examples of cosolvents of interest include, but are not limited to: n-methyl-2-pyrrolidone, deet, ethyl alcohol, methyl alcohol, polyethylene glycol (e.g., low molecular weight polyethylene glycol, such as PEG 600 or lower, e.g., 500, 400, 300, 200, 100 etc. and blends) thereof, and isopropyl myristate, etc. The cosolvent may be made up of a simple component or be in combination of two or more components.

Furthermore, in addition to the aforementioned ingredients, various additives that are used in ordinary topical water-soluble patch preparations may be suitably compounded as needed, including inorganic substances such as kaolin, bentonite, and titanium dioxide; preservatives such as paraben; anionic, cationic, and nonionic surfactants; metallic aluminum crosslinking agents such as aluminum chloride, dried aluminum hydroxide gel, and dihydroxyaluminum aminoacetate; oils such as jojoba oil and castor oil; chelating agents such as EDTA; pH regulators such as malic acid, tartaric acid, and diisopropanolamine; alcohols such as ethanol; moisture retaining agents such as hyaluronic acid, aloe extract, and urea; perfumes; and coloring agents. The pH of the gel base composition typically is one that lies in a physiologically acceptable range, where the pH typically may range from about 4.0 to 7.0.

As mentioned above, the adhesive gel base 30 is typically present on a support 32. Support 32 is generally made of a flexible material which is capable of fitting as the subject's body moves and includes, for example, various non-woven fabrics, woven fabrics, spandex, flannel, or a laminate of these materials with polyethylene film, polyethylene glycol terephthalate film, polyvinyl chloride film, ethylene-vinyl acetate copolymer film, polyurethane film, and the like. The support 32 has a body-facing surface to which the gel base is applied, and an opposite garment-facing surface. One exemplary combination of an adhesive gel base and support 32 is shown and described in U.S. Pat. No. 8,105,624 issued to Shud, and is incorporated herein by reference.

In addition to the adhesive gel base 30 and the support 32, the subject topical patches may also include a release film 34 on the surface of the gel base 30 opposite the support 32. The release film 34 protects the gel layer from the environment. The release film 34 may be any convenient material, where representative release films include polyesters, such as PET or PP, and the like.

Prior to use, patch 22 may be stored in a sealed package (not shown). Generally, the sealed package is fabricated from a packaging material that includes a layer made out of a material capable of preventing passage of moisture, oxygen and other agents, i.e., the package includes a moisture/oxygen barrier material. Any suitable barrier material may be employed, where barrier materials of interest include metallic layers, e.g., aluminum, where in many embodiments, the barrier layer is an aluminum layer. This barrier layer has a thickness sufficient to provide for the barrier function, where the thickness typically ranges from about 5 to 15 μm, usually from about 6 to 10 μm. In many embodiments, the package is a laminate of the barrier layer in combination with one or more additional layers, e.g., polymeric layers, paper layers, etc.

The topical patch 22 may be fabricated using any convenient method. One exemplary method for fabrication of the patches 22 includes preparing an adhesive gel paste through the uniform mixing of the aforementioned ingredients and then coating the paste onto the support 32, followed by cutting of the resultant product to the specified size to obtain the desired patch 22. For protection, the surface thereof is then covered with a release film of a polyester such as PET or PP.

The resultant topical patch 22 is then housed in a sealed package. Typically there is one patch 22 to a package. In one embodiment, the packaging material may contain an aluminum layer as a barrier to the external environment. For one possible fabrication protocol, see U.S. Pat. No. 5,827,529; the disclosure of which is herein incorporated by reference.

Patch 22 is self-adhesive, i.e., inherently adhesive, and thus may be fixed in a position over a target area of the skin, i.e., removably bonded to a desired target, without the use of additional adhesives or other means to hold the patch in place. For example, the composition of the adhesive gel base 30 may itself be adhesive.

In other embodiments, the patch 22 may be held in a fixed position on the target area of the skin using a separate adhesive such as an adhesive backing or the like, as seen in FIG. 2B. This adhesive is applied to the support perimeter which is in the area surrounding the adhesive gel base. In yet another embodiment, a combination of inherent adhesiveness and an additional separate adhesion means may be employed.

Certain adhesive gel compositions may be adapted and employed for use with the subject invention. Representative hydrogel patch compositions that may be adapted for use with the subject invention include, but are not limited to those described in U.S. Pat. Nos.: 5,120,544; 5,160,328; 5,270,358; 5,423,737; 5,476,443; 5,489,262; 5,501,661; 5,827,529; 6,039,940; 6,096,333; 6,214,374; 6,296,869; 6,348,212; 6,455,065; the disclosures of which are herein incorporated by reference.

As mentioned, once the adhesive gel paste is applied to the support 32, the resulting material is cut to the specified size to obtain the desired patch 22. The shape of patch 22 may vary, where representative shapes include square, rectangle, oval, circle, etc. The size of patch 22 may also vary, where in many embodiments the size ranges from about 1 cm², or about 15 cm², or about 30 cm², or about 40 cm².

It should be noted that the above manufacturing methods are merely representative. Any convenient method that is capable of producing the patch, as described above, may be employed.

### Carrying Case and Container

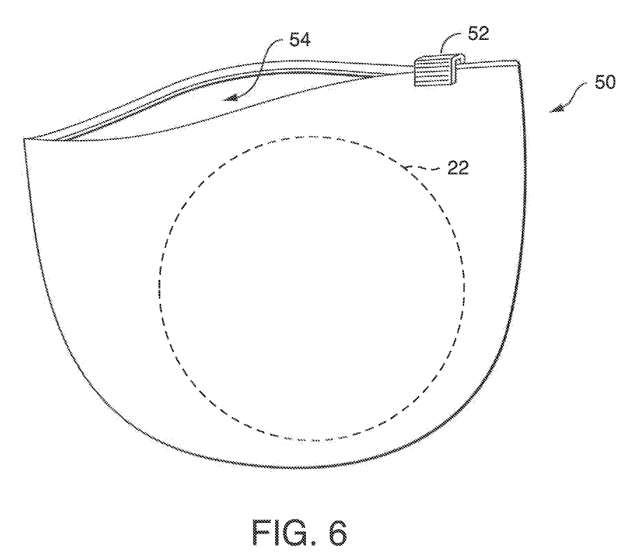

According to one aspect of the present disclosure, the carrying case may be a simple pouch 50 as seen in FIG. 6. Pouch 50 may be made from flexible, relatively inexpensive plastic or nonwoven materials, each of which is very practical for disposal after only one or a few uses. Pouch 50 may also be made from more durable flexible materials such as woven fabrics, leather, vinyl, or other materials which are more suitable for use with refill patches 22. Pouch 50 may have a zipper enclosure 52 as shown. However, it is contemplated that pouch 50 may have a flap closure with a fastener (pin, snap, hook and loop, hook and eye, button, or the like) to fasten the flap to a side of the pouch 50. Desirably, pouch 50 has a smooth interior surface 54 so that the adhesive gel remains as smooth as possible and does not pick up lint or dust which would negatively affect the adhesive quality of the adhesive gel. Further, pouch 50 has the characteristic of being water resistant and air impermeable to keep the adhesive gel from drying out. Many other embodiments are possible, each having the characteristic of protecting patch 22, either before or between uses, from being abraded, dried out, or from the premature depletion of any volatile stimulation agents.

According to another aspect of the present disclosure, a container 60 may be used to contain one or more packaged patches 22 and possibly all other kit 20 components. Container 60 may be made from plastic, wood, metal or other rigid materials.

### Method of Use

The patch 22 may be administered to a target area of the body having receptors innervated by the larger cutaneous nerve fibers (i.e. Aβ, Aδ). These sensory fibers are responsible for the sub-modalities of low-threshold touch, pressure, cold and pain. In the present disclosure, areas that are innervated by targeted receptors and neural pathways are located at the lower back, buttocks and genitals. Cutaneous nerves that host the sensory receptors and transmit inputs to the spinal cord include:

1. Superior cluneal nerve (arise from L1, L2, L3 (not shown));

2. Medial cluneal nerve (arise from S1, S2, S3);

3. Inferior cluneal nerve (posterior cutaneous femoral nerve; arises from S1, S2, S3);

4. Perforating cutaneous nerve (arises from S1, S2, S3);

5. Pudendal nerve (arises from S2, S3, S4).

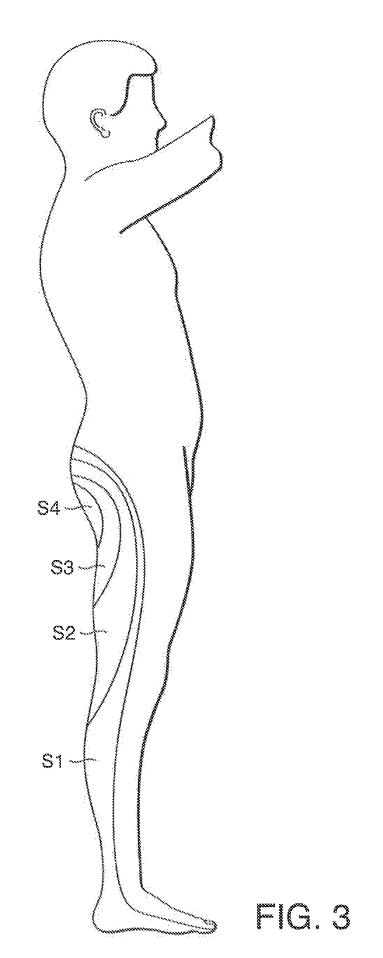

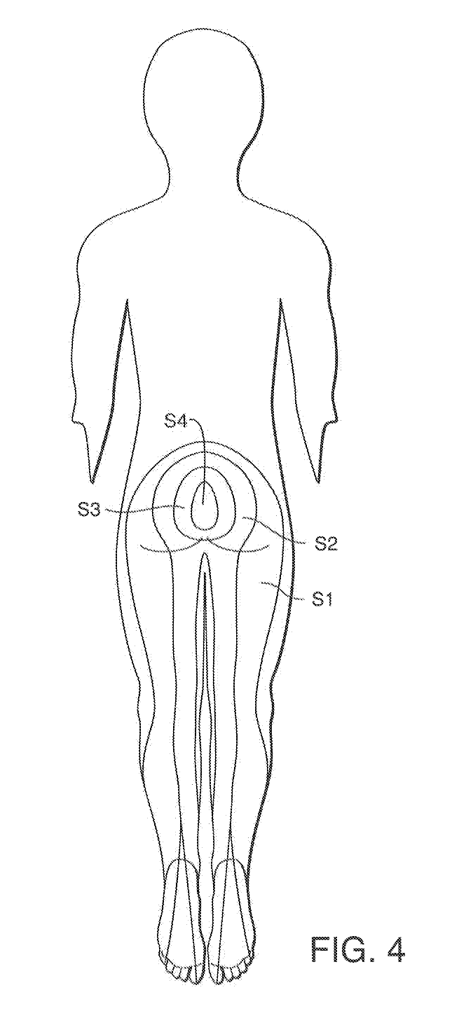

See FIGS. 3 and 4 showing the S1 through S4 dermatomes which are target areas, with the S3 dermatome being the most desirable target area.

The body region that is covered by the topical patch 22 following application is sufficient to provide for the desired amount of stimulation. For example, patch 22 need not cover all the aforementioned dermatomes. It is adequate and desirable to cover only a portion of one dermatome. Desirably, the area of stimulation where the patch 22 may be placed is about 36 to about 42 cm², or about 39 to about 41 cm², or about 40 cm².

The period of time between stimulus application and bladder inhibition is on the order of seconds to minutes, and the inhibitory effects will last as long as the patch continues to deliver the active ingredient to the skin. Once the patch is removed from the skin, the bladder is no longer inhibited, and normal bladder functions can resume.

In practice, patch 22 may be applied a single time or a plurality of times over a given time period. For example, one may decide to leave the patch 22 on site for two hours, remove it for 20 minutes, and replace it for another hour. In representative embodiments, the method is used in the treatment of a disease condition. By treatment it is meant that at least an alleviation of the symptoms associated with the pathological condition afflicting the subject, where alleviation is used in a broad sense to refer to at least a reduction in the magnitude of the urge to urinate. As such, treatment also includes situations where the pathological condition, or at least symptoms associated therewith, are completely inhibited, e.g. prevented from happening, or stopped, e.g. terminated, such that the subject no longer suffers from the premature urge to urinate, or at least the symptoms that characterize the pathological condition.

### Garment

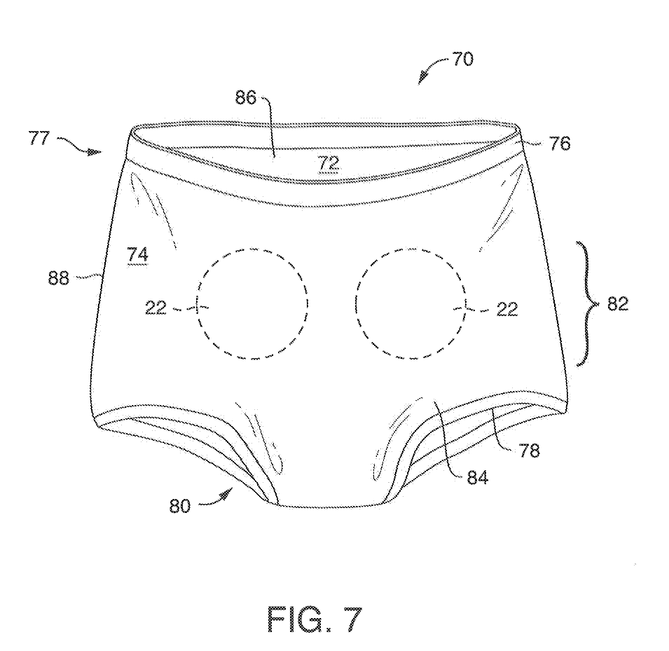

Referring now to FIG. 7, shown is one exemplary garment 70 in the form of a pant, similar to a typical pair of underwear. Garment 70 has a body-facing surface 72 and an exterior surface 74. Further, garment 70 has a waist region with a waist band 76. Opposite the waist region is a pair of leg bands 78 adjacent the crotch region 80. Between the waist region and the crotch region is the mid-region 82. Garment 70 has a rear panel 86 and a front panel 84 connected at sides 88.

Garment 70 may be made from various fabrics that are practical to dispose of after only one day of use. These fabrics may be non-woven fabrics, which may have the characteristic of being breathable.

In one embodiment, the garment 70 is made partially from elastomeric material so the pant hugs the body. For example, the front panel 84 may be elastomeric and the rear panel 86 non-elastomeric. In the alternative, there may be elastomeric gussets (not shown) at the sides 88 connecting the front panel 84 to the rear panel 86.

In one embodiment, a dosage of stimulation composition is applied to the body-facing surface 72 of the back panel 86 in the mid-region 82. Desirably, a non-breathable support is positioned between the stimulation composition and the garment fabric. For example, the patch 22 as described herein may be attached to the body-facing surface 72 by means of an adhesive, thermal bonding, stitching or the like. The support 32 is attached to the body-facing surface so that the adhesive gel 30 is exposed to the skin of the wearer of garment 70.

It may be desirable to apply the stimulation composition to the garment 70 bilaterally so that the sensory receptors of each buttock are stimulated. The stimulation composition is positioned so that when the garment 70 is worn, it covers a sacral dermatome, preferably S3.

### Instructions

One exemplary method for using patches 22 bilaterally is as follows:

1. Separate the protective backing from the patch.

2. Apply the ‘sticky’ side of the patch to a buttock, at a site approximately 2 to 3 finger breadths lateral to the tip of the tailbone, and 3 to 4 finger breadths superiorly.

3. Apply the second patch to the same site on the other buttock.

### EXAMPLES

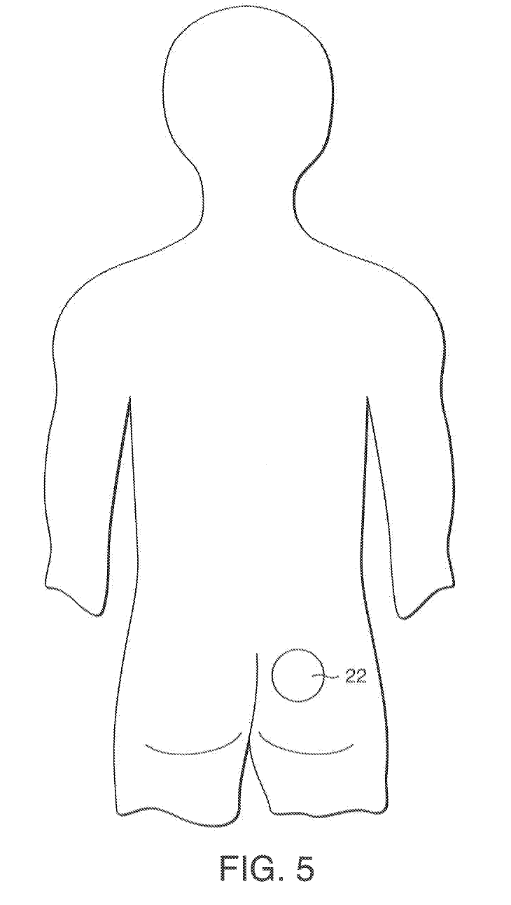

Subjects were instructed to apply a cold stimulus to their skin upon feeling the urge to void, and then to report their sensations. Subjects were given the choice of either BIOFREEZE, available from The Hygenic Corporation, Akron, OH; or ice. Both stimuli are known to activate the Aδ fibers. Stimuli were applied to skin regions bilaterally and covered the S1-S4 dermatomes at mid-buttocks (FIG. 5). The stimulation area was circular in shape and measured approximately 7 cm in diameter. The area's center was found by palpating the coccyx (tip of the tailbone) and moving 3-4 finger widths superiorly (towards the head) and 2-3 finger widths laterally.

Three of the four participants applied BIOFREEZE and one applied an ice pack upon feeling the urge to urinate. All participants reported a decrease in the urge to urinate within 2 minutes. The urge to urinate was abolished in persons who applied the BIOFREEZE for durations ranging from 27 to 105 minutes. The urge to urinate was similarly abolished in a subject that applied the ice pack. The ice pack was applied for 4 to 5 minutes, and the urge returned within 2 minutes of removing it from the skin.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

1. A kit for the alleviation of a subject's urge to urinate comprising:
a support having a body-facing surface and a garment-facing surface;
a composition comprising a stimulation agent selected from the group consisting of menthol, a menthol derivative, and capsaicin; disposed onto or for disposition by the subject onto an area of the body-facing surface; wherein the stimulation agent is in an amount that can effectively modulate sensory receptors in cutaneous nerves or nerve branches to temporarily alleviate the urge to urinate; and
a set of instructions regarding the placement of the composition onto the subject's buttock.

2. The kit of claim 1, wherein the support is a wipe substrate.

3. The kit of claim 1, wherein the support is a patch.

4. The kit of claim 3, wherein the support has an application area on the body-facing surface to which the composition is applied, and a perimeter surrounding the application area to which an adhesive is disposed.

5. The kit of claim 3, wherein the support has an application area between about 1 cm² and 41 cm².

6. The kit of claim 1, wherein the menthol derivatives are selected from the group consisting of menthyl lactate, menthoxypropandiol, menthone glycerine acetal, isopulegol and combinations thereof.

7. A method for mitigating a subject's urge to urinate comprising the steps of:
providing the subject with a support having a body-facing surface and a composition thereon, the composition comprising a stimulation agent selected from the group consisting of menthol, menthol derivatives and capsaicin, wherein the stimulation agent is in an amount that can effectively modulate sensory receptors in cutaneous nerves or nerve branches to temporarily alleviate the urge to urinate; and
providing instruction as to the placement of the support on a target area of the subject.

8. The method of claim 7, wherein the stimulation agent consists of menthol menthol derivatives, or a combination thereof.

9. The method of claim 7, wherein the target area of the subject comprises an area covering a portion of the subject's S1-S4 dermatomes.

10. The method of any one of claim 7, wherein the target area is about 36 to 42 cm².

11. The method of claim 7, wherein the target area comprises a portion of the S3 dermatome.

12. The method of claim 7, wherein the target area is on the subject's buttock.

13. A disposable pant comprising:
a body-facing surface and an opposite exterior surface; a front side, a back side and a crotch region;
wherein outside of the crotch region, the body-facing surface of the back side has a composition disposed thereon, the composition comprising menthol, menthol derivatives, capsaicin, or a combination thereof.

14. The disposable pant of claim 13, wherein the pant comprises a nonwoven fabric.

15. The disposable pant of claim 13, wherein the pant comprises an elastomeric fabric.

16. The disposable pant of claim 13, further comprising a liquid impermeable support between the composition and the body-facing surface of the back side.
